(19) United States
(12) Patent Application Publication
(10) Pub. No.: US 2026/0248708 A1
(43) Pub. Date: Aug. 27, 2026

(54) SKIN ANTI-AGEING COMPOSITION

(71) Applicant: LYMA LIFE LIMITED, London (GB)

(72) Inventor: Lucy GOFF, London (GB)

(21) Appl. No.: 19/489,702

(22) PCT Filed: Jun. 5, 2024
(86) PCT No.: PCT/EP2024/065499

§ 371 Date: Dec. 3, 2025

(30) Foreign Application Priority Data
Jun. 5, 2023 (GB) 2308387.6

(51) Int. Cl. A61K 8/67; A61K 8/34; A61K 8/49; A61K 8/73; A61K 8/92; A61K 8/9794; A61Q 19/08

(52) U.S. Cl. CPC A61K 8/671; A61K 8/345; A61K 8/498; A61K 8/73; A61K 8/922; A61K 8/9794; A61Q 19/08

(57) ABSTRACT

This invention relates to a cosmetic composition. The cosmetic composition is a complex combination product. The cosmetic composition is an anti-ageing composition. The product has been engineered to include a high number of active ingredients. The composition comprises retinyl palmitate, Silybum marianum seed oil, betaine, ergothioneine, yeast beta-glucan, Triticum vulgare germ extract, quercetin, and propanediol.

This invention relates to a cosmetic composition. More specifically, the present invention relates to a skincare composition that has an anti-aging effect on the skin, in particular the face. The composition may be a two-part composition with a cream and a serum. The present invention also contemplates a kit comprising the cream and the serum. Also contemplated are methods of using the composition of the present invention.

### BACKGROUND

Skin care compositions have been present since the dawn of recorded history. With the ancient Egyptians utilising naturally occurring items such as olive oil and essential oils to attend to skin conditions. In the past 40 to 50 years, the cosmetics industry has significantly expanded their skin care offerings with compositions aimed at a multitude of different effects, such as ageing, hydration, skin elasticity and many others. However, not until more recently has the skin care industry begun to understand the science of the skin.

As the science of ageing has developed it has become apparent that the overall process of ageing is driven by multiple sub-routines, and that many of these sub-routines are malleable. One example of this relates to the ageing of skin. The well-known macroscopic changes including wrinkles, skin discoloration, skin stretching, etc, are driven by micro-structural changes in the extra-cellular matrix (ECM), micro-circulatory, mitochondrial and immunological decline, epigenetic shift, the related accumulation of senescent cells and time-related changes in the dermal and epidermal microbiota. Chronic inflammatory stress is recognised as playing an important role in many of these sub-routines, as is nutritional status. We generally refer to these processes as mechanisms of ageing.

Current topical skincare protocols are marketing rather than evidence lead, formulations comprised of amino acids with under dosed dipeptides, tripeptides and oligopeptides. Small amino acid chains (tripeptides, oligopeptides) have shown great promise in tissue regeneration because they can mimic some of the actions of the growth factors (which are much larger molecules) while being small enough to be absorbed through the skin. Hence, they can be delivered topically. However, if they are underdosed, the product will not be efficacious. The right formulation of emollients, humectants and skin conditioners with clinically effective oligopeptides would be a superior option, but this does not exist.

Therefore, it is an aim of certain embodiments of the present invention to provide a composition comprising clinically effective amounts of active agents, for example, oligopeptides. However, the greater number of components within the composition, such as oligopeptides, the greater the difficulty to create a formulation that meets the specifications for a skin care composition. Therefore, it is also an aim of the present composition to provide a therapeutically effective number of active agents to the skin.

More generally, if the number of ingredients within a composition are increased (which are water soluble or lipid soluble) it is increasingly difficult to produce a stable product. A composition comprising a large number of components are normally divided into separate formulas. An aim of the present invention is to provide a composition with 2 separate compositions comprising a large number of skin active ingredients.

The advent of an understanding of the skin microbiome along with a better characterisation of the mechanisms that result in ageing skin have led to the provision of more effective skin care compositions. Following from the understanding of the ageing mechanisms within the skin, skin care therapies have begun targeting specific mechanisms. In many cases, skin care therapies have begun targeting multiple mechanisms of skin ageing. However, targeting multiple mechanisms of ageing results in complications. Targeting multiple mechanisms of ageing requires the inclusion of multiple active agents, which results in challenges to create compositions due to incompatibilities or differing requirements of the active agents along with the aesthetic and physical requirements of a skin care composition. Therefore, it has proven challenging to formulate skin care compositions capable of targeting multiple causes of skin ageing because of the requirement for all actives to be stable, bioavailable and be delivered to the layer of the skin where they are required.

Prior art formulations are generally straightforward, low-tech compositions. The formulations follow a general template based on building extra cellular matrix and hydration. Certain prior art formulations tend to have fewer active ingredients and have less combinations of ingredients. Prior art compositions also rely on silicone-based ingredients rather than plant extracts. However, even formulations suggested to be more therapeutic in nature generally supplement plant-based ingredients with synthetic oils and bulking agents.

Prior art composition have a large number of ingredients, sometimes in the region of 25-50 ingredients. When applying a relatively small amount of a composition to the skin the amounts of each of those ingredients will have very little effect. This is exacerbated by prior art skin care compositions generally including around 80% of ingredients that are directed to the improvement of skin appearance with 20% of the ingredients being directed to rebuilding the skin. In contrast, the present invention comprises a much greater percentage of ingredients aimed to rebuild the skin.

The present invention, in certain embodiments, aims to target the causes of ageing rather than simply addressing signs of ageing. Thus, the present invention aims to rebuild the skin.

Common ingredients within skin care products are retinol and vitamin C based. However, these components have a very superficial act on skin ageing, being unable to get to the heart of skin reprogramming and affect skin ageing.

Prior art skin care compositions are heavily dominated with products that benefit only a few mechanisms of skin ageing; generally moisturising, botanical extracts with scientific justification, pro-anabolic and anti-catabolic benefits. This approach, targeting four mechanisms of ageing, is superficial. The ageing process, being truly multifaceted, involves at least ten mechanisms which all contribute to skin ageing.

The ten mechanisms of skin ageing are:
1. microbiome shift;
2. hydration failure;
3. microcirculatory collapse;
4. immunological rundown;
5. senescence;
6. mitochondrial decline;
7. epigenetic shift;
8. pro-anabolic with respect to the extra cellular matrix;
9. anti-catabolic with respect to the extra cellular matrix; and
10. anti-inflammatory

Accordingly, it is an aim of the present invention to provide a composition comprising multiple ingredients capable of targeting multiple mechanisms of ageing in a stable composition. In particularly preferred embodiments, the present invention aims to target ten mechanisms of skin ageing. It is also an aim of the present invention to provide a composition containing multiple active agents that is stable. The present invention additionally or alternatively aims to provide certain embodiments that provide bioavailable active agents that are optionally delivered to the layer of the skin where they are required.

The mechanisms of ageing that are considered herein are discussed in further detail below.

### Microbiological Shift

The skin has its own microbiota, and as we get older the dermal population of microbes changes in ways that lead to a run-down of skin repair, enhanced inflammation and accelerates matrix breakdown. There is good evidence to suggest that active agents can modulate the skin microbiome to encourage a more youthful microbiome—and more youthful looking skin.

### Hydration Failure

Skin tends to dry out as we get older. There are several reasons for this, but the major cause is degradation of the skin's extracellular matrix. The extracellular matrix acts like a sponge, capturing and maintaining moisture within the skin. However, as the extracellular matrix degrades the internal spaces, where the water is held degrade and are lost. Therefore, the skin simply cannot hold as much fluid. The water retention, consequently hydration, can be boosted by re-building the extra-cellular matrix.

### Microcirculatory Collapse

We become more vulnerable to circulatory disease as we age, and this circulatory deterioration also occurs in the skin. As such, the ability of our skin cells to gain the nutrients and oxygen they require is diminished and skin cells become less able to remove waste products. This shows on the skin. Stabilising the capillary beds in the skin improves the microcirculation and repairs skin.

### Immunological Rundown

There is convincing evidence that older skin is less immuno-competent. This means that the skin's own immune cells (such as Langerhans cells) become less able to defend against infections and other fundamental cellular errors. Potent immuno-modulators have been shown to enhance innate immune function.

### Senescence

Senescence is the body's method of removing old cells. Most cells within a body have a lifetime because cells do start to wear out. When cells have gotten to the point where they are not able to properly function the cells should shut themselves off. This is because they have become a burden to the tissue as a whole. Most ageing cells do as they should, However, some cells remain past there functioning lifetime, using up resources and triggering chronic inflammation. This ages the skin dramatically. These cells are called senescent cells and need to be removed to rejuvenate the skin.

### Matrix Decay

The skin matrix is a very dynamic tissue and is very good at healing itself—which is, fundamentally, how a cut heals. With the passage of time, the rate of matrix decay tends to accelerate and the rate of matrix repair slows, so the amount of matrix is progressively reduced. Inevitably dryness, sagginess and wrinkles start to form.

### Mitochondrial Decline

Skin cells become less able to generate energy as they age. To put this simply, your skin gets tired. It is possible to “re-set” the mitochondrial clock and give your cells an energy boost, rejuvenating your skin to make the skin look visibly younger.

### Epigenetic Drift

This describes changes in the ‘epigenetic clock’, an intra-cellular modulator of cell functions which regulates which genes are active at any one time, and which ones are quiescent. As we age, it is common to lose methyl groups on your DNA, and acetyl groups on your histones, proteins which protect and regulate DNA functions. Retaining the methyl groups and the acetyl groups has a positive effect on epigenetic drift, reverting the skin cells to a younger state.

One mechanism by which the present invention rebuilds the skin is by targeting the skins extracellular matrix (ECM). The ECM is constantly being broken down and repaired. When the rate of ECM breakdown is greater than the rate of repair the skin ages. Conversely, when the rate of ECM repair is greater than the rate of breakdown then skin aging can be reversed. In certain embodiments the present invention aims to promote repair of the ECM and/or prevent the breakdown of the ECM.

### BRIEF SUMMARY OF THE DISCLOSURE

In accordance with the present invention there is provided a composition comprising retinyl palmitate, Silybum marianum seed oil, betaine, ergothioneine, yeast beta-glucan, Triticum vulgare germ extract, quercetin, and propanediol.

In certain embodiments the composition comprises the active ingredients as commercially available products. As such, the composition of the present invention may comprise: retinyl palmitate, Silybum marianum seed oil (milk thistle oil), Genencare® OSMS BA (betaine), MitoPrime™ (ergothioneine), Wellmune® (yeast beta-glucan), Triticum vulgare germ extract, Quercevita™ (quercetin), and Pro-Lipo™ Neo (propanediol) and/or Zemea® (propanediol).

In certain embodiments the retinyl palmitate is present in an amount of from about 0.25 to about 0.5 wt %. Preferably, the retinyl palmitate is present in an amount of about 0.35 wt %.

In certain embodiments, Silybum marianum seed oil (milk thistle oil) is present in an amount of from about 1 to about 3 wt %. Preferably, the Silybum marianum seed oil is present in an amount of about 2 wt %.

In certain embodiments the betaine is present in an amount of from about 2 to about 4 wt %. Preferably, the betaine is present in an amount of about 2.5 wt %.

In certain embodiments the ergothioneine is present in an amount of from about 0.01 to about 0.1 wt %. Preferably, the ergothioneine is present in an amount of about 0.05 wt %.

In certain embodiments the yeast beta-glucan is present in an amount of from about 0.05 to about 0.3 wt %. Preferably, the yeast beta-glucan is present in an amount of about 0.1 wt %.

In certain embodiments the Triticum vulgare germ extract is present in an amount of from about 0.1 to about 3 wt %. Preferably, the Triticum vulgare germ extract is present in an amount of about 1 wt %.

In certain embodiments the quercetin is present in an amount of from about 0.002 to about 0.4 wt %. Preferably, the quercetin is present in an amount of about 0.1 wt %. In embodiments the quercetin is present within the composition as the commercially available product Quercevita™. Accordingly, in certain embodiments the composition may comprise Quercevita™ in an amount of from about 0.01 to about 2 wt %, preferably in an amount of about 0.5 wt %.

In certain embodiments the propanediol is present in an amount of from about 1 to about 8 wt % or from about 3 to 8 wt %. Preferably, the propanediol is present in an amount of about 5.5 or about 6.2 wt %. In embodiments the propanediol is present within the composition as the commercially available Pro-Lipo™ Neo and/or Zemea®. Accordingly, in certain embodiments the composition comprises Pro-Lipo™ Neo and/or Zemea® in a total amount of from about 1 to about 10 wt % or from about 4 to about 10 wt %, preferably in an amount of about 7 or about 8 wt %.

In certain embodiments, the composition may further comprise one or more excipients selected from: a solvent, an emulsifier; an emollient; a humectant; a skin conditioner; an exfoliant; a cleansing agent; an immune response modifier; an anti-inflammatory; a pigment balancer; an antioxidant; a retinoid; a sebum regulator: a phytoestrogen; an amino acid; a matrikine peptide; a preservative; a thickener; a stabiliser; a pH balancer; a film forming agent; and a bulking agent.

In embodiments the composition may additionally comprise one or more components selected from: Jojoba oil, macadamia seed oil esters, squalene, phytosteryl macadamiate, phytosterols (L22), cetearyl alcohol, glyceryl stearate, jojoba esters, Helianthus annus (sunflower) seed wax, sodium stearoyl glutamate, polyglycerin-3 (Emulium Dolcea®), palmitamide MEA (Levagen®), phenetyl alcohol, planktonic exopolysaccharide 3 (EPS Seafill), tocopherol, Zizania latifolia stem extract, lecithin, (Pro-Lipo™ Neo) heptyl undecylenate, and C₁₃₋₁₅ alkane (LexFeel™ WOW-A DT).

In embodiments the composition may further comprise one or more components selected from: sodium ascorbyl phosphate (Stay-C® 50), Simmondsia chinensis (jojoba) seed oil, Olea europaea (olive) leaf extract, sodium lactate, Sodium pyrrolidone carboxylic acid, fructose, glycine, inositol, niacinamide, urea, sodium benzoate, lactic acid (Lactil®), Avena sativa kernel extract, Lactobacillus ferment (aurafirm), Acetyl tetrapeptide-40, Caprylyl glycol (Telangyn™), rutin, and Undaria pinnatifida extract (Ephemer™).

In embodiments the composition may further comprise one or more components selected from: Centaurea cyanus flower water, Butyrospermum parkii butter, Xylitylglucoside, Anhydroxylitol, Xylitol (Aquaxyl™), Astrocaryum murumuru seed butter, bakuchiol, Palmitoyl glycine (Timecode™), Acetyl octapeptide-3, Magnesium aspartate, Zinc gluconate, Copper gluconate (Sepitonic™ M4), Glycerin, Cucurbitaceae (gourd) extract, Lactobacillus/brassica nigra seed ferment extract, lactococcus ferment extract, Pentylene glycol (Wonderage™), Hydrolysed hyaluronic acid (PrimaHyal™ 50), Bisabolol and cetearyl alcohol, cetearyl glucoside, glucose (Montanov™ 68).

In certain embodiments the composition comprises water. Where the composition is described as comprising various components in a particular amount, the balance of the composition may be water.

In certain embodiments the composition is a cream. In alternative embodiments the composition is a serum. In preferred embodiments the composition is a two-part composition with a first composition and a second composition, wherein the first composition comprises retinyl palmitate, Silybum marianum seed oil, betaine, ergothioneine, yeast beta-glucan, Triticum vulgare germ extract, quercetin, and propanediol, and the second composition comprises retinyl palmitate, Silybum marianum seed oil, betaine, ergothioneine, yeast beta-glucan, Triticum vulgare germ extract, quercetin, and propanediol.

In certain embodiments the first composition and the second composition have different viscosities. For example, the first composition will have a lower viscosity than the second composition. In certain embodiments the first composition is a serum and the second composition is a cream.

In certain embodiments the first composition and the second composition contain different amounts of retinyl palmitate, Silybum marianum seed oil, betaine, ergothioneine, yeast beta-glucan, Triticum vulgare germ extract, quercetin, and propanediol (collectively called the components along with any other ingredient contained within the composition of the invention). In certain embodiments the first composition and the second composition contain the same amounts of the components. As would be expected by the skilled person, the first composition and the second composition may contain certain components with the same amount and other components with a different amount.

In certain embodiments the amount of retinyl palmitate, Silybum marianum seed oil, betaine, ergothioneine, yeast beta-glucan, Triticum vulgare germ extract, and quercetin is the same in the first composition and the second composition and the amount of propanediol is different.

In certain embodiments the composition comprises retinyl palmitate in an amount of from about 0.25 to about 0.5 wt %, Silybum marianum seed oil (milk thistle oil) in an amount of from about 1 to about 3 wt %, betaine in an amount of from about 2 to about 4 wt %, ergothioneine in an amount of from about 0.01 to about 0.1 wt %, yeast beta-glucan in an amount of from about 0.05 to about 0.3 wt %, Triticum vulgare germ extract in an amount of from about 0.1 to about 3 wt %, quercetin in an amount of from about 0.002 to about 0.4 wt % (optionally the quercetin may be comprised in the composition as Quercevita™ in an amount of from about 0.01 to about 2 wt %), and propanediol in an amount of from about 1 to about 8 wt % or about 3 to about 8 wt % (optionally the propanediol may be comprised in the composition as Pro-Lipo™ Neo and/or Zemea® in an amount of from about 1 to about 10 wt % or about 4 to about 10 wt %).

In certain embodiments, the retinyl palmitate is present in an amount of about 0.35 wt, the Silybum marianum seed oil is present in an amount of about 2 wt %, the betaine is present in an amount of about 2.5 wt %, the ergothioneine is present in an amount of about 0.05 wt %, the yeast beta-glucan is present in an amount of about 0.1 wt %, the Triticum vulgare germ extract is present in an amount of about 1 wt %, the quercetin is present in an amount of about 0.1 wt % (or Quercevita™ is present in an amount of about 0.5 wt %, the propanediol (or Pro-Lipo™ Neo and/or Zemea®) is present in an amount of about 5.5 or about 6.2 wt % (or Pro-Lipo™ Neo and/or Zemea® is present in an amount of about 7 or about 8 wt %). The propanediol may be present in the first composition in an amount of about 5.5 wt % (or Pro-Lipo™ Neo and/or Zemea® may be present in the first composition in an amount of about 7 wt %). The propanediol (Pro-Lipo™ Neo and/or Zemea®) may be present in the second composition in an amount of about 6.2 wt % (or Pro-Lipo™ Neo and/or Zemea® may be present in the first composition in an amount of about 8 wt %).

In certain embodiments of the present invention, the composition further comprises thyme oil. In certain embodiments the composition further comprises magnolia officials bark extract, thymus vulgars (thyme) leaf oil, and glyceryl laurate.

In certain embodiments the composition further comprises Centaurea cyanus flower water. Centaurea cyanus flower water provides beneficial effects over the use of water as the aqueous phase. Centaurea cyanus flower water not only provides a liquid aqueous medium but it also provides anti-inflammatory and skin soothing properties.

In certain embodiments the quercetin is incorporated into the composition as a mixture with lecithin. Accordingly, in certain embodiments the composition further comprises lecithin. In certain embodiments the quercetin is present in the composition in an encapsulated form. This has the benefit of providing stable quercetin. Furthermore, this has the benefit of avoiding any potential discolouration that could be caused by quercetin.

In certain embodiments the composition comprises a plurality of liposomes. In certain embodiments the composition comprises liposomes that are adapted to deliver lipophilic and hydrophilic molecules.

In certain embodiments the liposomes comprise propanediol, lecithin, tocopherol and Helianthus annuus (sunflower seed oil). The liposomes may be formed from propanediol, lecithin, tocopherol and Helianthus annuus (sunflower seed oil). The liposomes may be adapted to include both lipophilic and hydrophilic molecules. In certain embodiments the liposomes contain one or more components selected from: betaine, Silybum marianum seed oil, ergothioneine, Triticum vulgare germ extract, palmitamide MEA, quercetin, retinyl palmitate, yeast beta-glucan, and Zizania latifolia stem extract.

In certain embodiments the liposomes may be formed from propanediol, lecithin, tocopherol and Helianthus annuus (sunflower seed oil) and the liposomes contain betaine, Silybum marianum seed oil, ergothioneine, Triticum vulgare germ extract, palmitamide MEA, retinyl palmitate, yeast beta-glucan, and Zizania latifolia stem extract.

The presence of liposomes within the compositions greatly improves the stability of the composition compared to a composition where the components contained by the liposomes are free within the composition.

The components that are not present in the liposomes are introduced into the composition as a powder and are dissolved in water or milk thistle oil.

In a further aspect of the present invention there is provided a skin anti-ageing protocol wherein the protocol is a method of cosmetic therapy comprising application to a user's skin of:
a first composition comprising retinyl palmitate, Silybum marianum seed oil, betaine, ergothioneine, yeast beta-glucan, Triticum vulgare germ extract, quercetin, and propanediol; and
a second composition comprising retinyl palmitate, Silybum marianum seed oil, betaine, ergothioneine, yeast beta-glucan, Triticum vulgare germ extract, quercetin, and propanediol.

In embodiments the first composition is applied before the second composition. In embodiments the first composition is a serum and the second composition is a cream. In preferred embodiments of the protocol the cream is applied to the users skin after the serum.

In a further aspect of the present invention there is provided a kit comprising a first composition of the present invention and a second composition of the present invention.

The kit may further comprise instructions on how to apply the first composition and the second composition. The instructions may be details of how to carry out the method of the protocol of the present invention.

In certain embodiments the serum is a liquid and the cream has a higher viscosity than the serum. The serum may be contained in a pump operated bottle. The cream may be contained in a pot or some other lidded container. As such, the kit may comprise a pump operated bottle containing the first composition and a pot containing the second composition.

### DETAILED DESCRIPTION

The amounts components within the composition of the present invention are provided in weight percent (wt %). This weight percent is based on the total weight of the composition. The total weight of the composition includes any water contained within the composition. As such, the weight percent is based on a “wet” weight of the composition.

In embodiments the composition may further comprise acacia Senegal gum and xanthum gum (Solagum™ AX).

In embodiments the composition may further comprise hydroxyacetophenone (SymSave® H).

In embodiments the composition may comprise Jojoba oil, macadamia seed oil esters, squalene, phytosteryl macadamiate, and phytosterols (L22).

In embodiments the composition may comprise squalene.

In embodiments the composition may further comprise cetearyl alcohol, glyceryl stearate, jojoba esters, Helianthus annus (sunflower) seed wax, sodium stearoyl glutamate, polyglycerin-3 and tocopherol (Emulium Dolcea®).

In embodiments the composition may further comprise palmitamide MEA (Levagen®).

In embodiments the composition may further comprise phenetyl alcohol and planktonic exopolysaccharide 3 (EPS Seafill).

In embodiments the composition may further comprise Tocopherol

In embodiments the composition may further comprise Zizania latifolia stem extract.

In embodiments the composition may further comprise lecithin, (Pro-Lipo™ Neo)

In embodiments the composition may further comprise heptyl undecylenate, and C₁₃₋₁₅ alkane (LexFeel™ WOW-A DT).

In embodiments the first composition may further comprise Simmondsia chinensis (jojoba) seed oil.

In embodiments the first composition may further comprise sodium ascorbyl phosphate (Stay-C® 50).

In embodiments the first composition may further comprise rutin.

In embodiments the first composition may further comprise sodium lactate, sodium pyrrolidone carboxylic acid, fructose, glycine, inositol, niacinamide, urea, sodium benzoate, lactic acid (Lactil®).

In embodiments the first composition may further comprise Acetyl tetrapeptide-40, Caprylyl glycol (Telangyn™).

In embodiments the first composition may further comprise Olea europaea (olive) leaf extract.

In embodiments the first composition may further comprise Lactobacillus ferment, Avena sativa kernel extract, sodium benzoate and potassium sorbate (aurafirm P).

In embodiments the first composition may further comprise Caprylic/capric triglyceride and Undaria pinnatifida extract (Ephemer™).

In embodiments the second composition may further comprise cetearyl alcohol, cetearyl glucoside, and glucose (Montanov™ 68).

In embodiments the second composition may further comprise Centaurea cyanus flower water.

In embodiments the second composition may further comprise Butyrospermum parkii butter.

In embodiments the second composition may further comprise xylitylglucoside, anhydroxylitol, and xylitol glucose (Aquaxyl™).

In embodiments the second composition may further comprise Astrocaryum murumuru seed butter.

In embodiments the second composition may further comprise bakuchiol

In embodiments the second composition may further comprise Palmitoyl glycine (Timecode™).

In embodiments the second composition may further comprise acetyl octapeptide-3, and caprylyl glycol (SNAP-8™).

In embodiments the second composition may further comprise magnesium aspartate, zinc gluconate, copper gluconate and calcium gluconate (Sepitonic™ M4).

In embodiments the second composition may further comprise glycerin, cucurbitaceae (gourd) extract, lactobacillus/Brassica nigra seed ferment extract, lactococcus ferment extract and pentylene glycol (Wonderage™).

In embodiments the second composition may further comprise hydrolysed hyaluronic acid (PrimaHyal™ 50).

In embodiments the second composition may further comprise bisabolol.

In embodiments the second composition may further comprise cetearyl alcohol, cetearyl glucoside, and glucose (Montanov™ 68).

The solvent of the present invention may be an aqueous solvent or a lipid solvent. An aqueous solvent may be water. A lipid solvent may be selected from: ethyl oleate, ethyl linoleate, hexylresorcinol, propylene glycol, butylene glycol and any combination thereof.

The emulsifier of the present invention may be selected from: polyglyceryl-4-oleate, polyglyceryl-6-oleate, lecithin, C12, 20 Acid PEG-8, rice bran wax, beeswax, candelilla wax, polyacrylic acid and any combination thereof.

The emollient of the present invention may be selected from: Butyrospermum Parkii (Shea) butter, cocoa butter, coconut oil, almond oil, canola oil, avocado oil, rapeseed oil, jojoba oil, mango seed butter, argan oil, evening primrose oil, marula oil, grapeseed oil, hempseed oil, chia seed oil, apricot kernel oil, plum kernel oil, rosehip oil, sunflower seed oil, amaranthus seed extract, squalene and any combination thereof.

The humectant of the present invention may be selected from: hyaluronic acid, glycerine, glycerol, palmitoyl proline, β-glucans, chondroitin sulphate, allantoin and any combination thereof.

A skin conditioner according to the present invention is any compound that forms a protective layer preventing moisture loss. Specific skin conditioners that may be appropriate for the present invention may be selected from: Ceramides, glucosylceramides, salicylol phytosphringosene, sphringolipiden, glycosaminoglycans, inositol and combinations thereof.

An exfoliant of the present invention may be any compounds that enhances the natural epidermal turnover of the skin. The exfoliant may be selected from: alpha and beta hydroxyacids (AHA, BHA), tartaric acid, citric acid, urea and any combination thereof.

A cleansing agent is any excipient that cleans the skin of oxidised fatty acids which give a dirty appearance to the skin. Cleansing agent of the present invention may be selected from: Oleic acid, Lauric acid, PEG-12-Dimethicone and any combination thereof.

The immune response modifier and anti-inflammatory may be selected from: vitamin B3 (niacin) and (niacinamide), vitamin B5 (pantothenic acid), γ-linolenic acid, spirulina, evening primrose oil and combinations thereof.

A pigment balancer according to the present invention is any excipient that improves blotchy, unevenly pigmented skin. For example, the pigment balancer may be selected from: tranexamic acid, niacin (Vit B3), niacinamide (vit B5) and any combination thereof.

An antioxidant of the present invention is any compound that prevents oxidative damage to skin. The antioxidant may be selected from: vitamin C (L-ascorbic acid), Vitamin E (α-tocopherol), Q10, Green tea polyphenols, carotenoids, α-lipoic acid, flavonoids (optionally selected from resveratrol), ursolic acid, milk thistle extract and combinations thereof.

A retinoid of the present invention may be selected from: retinol, trans-retinoic acid (tretinoin), retinyl acetate, retinyl propionate, retinyl palmitate and any combination thereof.

A sebum regulator of the present invention may be selected from: retinols, Zinc-L-pyrrolidone carboxylic acid, niacin, niacinamide and any combination thereof.

A phytoestrogen of the present invention may be any plant based analogue of the estrogen hormone, such as isoflavones, e.g. soy isoflavanones.

An amino acid of the present invention may be selected from: glutamine, I-alanyl-1-glutamine, alanine, adenosine, I-arginine, phenylalanine or any combination thereof.

A matrikin peptide of the present invention is any peptide that induces collagen and extracellular matrix synthesis. The matrikin peptide may be selected from: oligopeptide 177, palmitoyl tripeptide-8, GHK-tripeptide, KTTKS-pentapeptide, acetyl hexapeptide-8, GEKG-tetrapeptide and combinations thereof.

A preservative of the present invention may be selected from: potassium sorbate, sodium benzoate, sodium citrate, sodium hexametaphosphate, hoelen, phytosphringosene, ethylhexylglycerine, disodium EDTA, parabens, phenoxyethanol; and combinations thereof.

A thickener or stabiliser may be selected from: xantham Gum, C13,14 isoparaffin, carob bean gum, glyceryl linolenate, sage leaf extract, algae extract and any combination thereof.

A pH balancer of the present invention may be selected from: cetyl phosphate, sodium hydroxide; and combinations thereof.

A film forming agent may be selected from: maltodextrin, sodium hyaluronate cross polymer, polyacrylamide and combinations thereof.

Triticum vulgare germ extract may also be known, and discussed herein, as spermidine.

Genencare® OSMS BA is a white crystalline powder marketed by Elemental and consists of betaine.

MitoPrime™ is an off white to white powder marketed by NNB Nutrition consists of ergothioneine.

L22® is a clear to pale yellow liquid with a melting point of approximately 5° C. marketed by FloraTech and comprises jojoba oil esters, macademia seed oil esters, squalene, phytosteryl macadamiate, phytosterols and tocopherol.

Emulium Dolcea® is a . . . marketed by Gattefosse and comprises cetearyl alcohol, glyceryl stearate, jojoba esters, Helianthus annus (sunflower) seed wax, sodium stearoyl glutamate, polyglycerin-3, tocopherol and water.

Levagen® is a white powder with a melting point of 96 to 100° C. marketed by Gencor comprising palmitamide MEA. Palmitamide MEA is a mixture of ethanolamides of palmitic acid. The composition of the present invention may be a composition prepared by a method that comprises dissolving Levagen® at a temperature of 90° C. If the Levagen® is dissolved at a temperature lower than 90° C. gritty particles may be present in the composition.

EPS Seafill is a colourless to lightly yellow liquid marketed by Codif International and comprises planktonic exopolysaccharide 3 (Alteromonas ferment extract), phenetyl alcohol and aqua.

Stay-C® 50 is a white/off-white powder with a melting point of greater than 200° C. COMPOSITION marketed by DSM consisting of sodium ascorbyl phosphate.

Lactil® is a yellowish liquid marketed by Evonik comprising sodium lactate, sodium pyrrolidone carboxylic acid, fructose, glycine, inositol, niacinamide, urea, sodium benzoate, lactic acid and aqua.

Aurafirm P is a viscous opaque liquid with a brownish-grey colouration marketed by Oat Services Ltd comprising sodium benzoate, potassium benzoate, Avena sativa kernel extract, Lactobacillus ferment and aqua.

Telangyn™ is a colourless liquid marketed by Lipotec comprising aqua, acetyl tetrapeptide-40 and caprylyl glycol.

Rutin is also called rutoside, quercetin-3-rutinoside, or sophorin it is a citrus flavonoid glycoside found in buckwheat.

Ephemer™ is an oily liquid with a yellow-green colour marketed by Seppic comprising caprylic/capric triglyceride and Undaria pinnatifida extract.

The composition of the present invention comprises one or more active ingredients that are powders when incorporated into the composition. The powder represents the most efficacious form of the active ingredient. Betaine, beta-glucan, ergothioneine and Zizania latifolia extract may be incorporated into the composition as a dissolved powder. However, use of a powder presents a problem as the powders must be dissolved to be incorporated into the composition. The present invention has addressed this problem by a method of dissolving the powders.

In certain embodiments the composition is a composition produced by a method comprising the step of dissolving one or more ingredients at a temperature of 40° C. and dissolving Levagen® at a temperature of 90° C. A composition formed by this method does not contain undesirable particulates.

Betaine, beta-glucan, ergothioneine and Zizania latifolia extract are dissolved within the composition at 40° C. The components are dissolved in either water or milk thistle oil. The hydrophilic components are dissolved in water. The lipophilic components are dissolved in milk thistle oil.

### Microbiological Shift

Spermidine has a beneficial effect on the skin microbiota. Spermidine has the effect of encouraging a more youthful microbiome by increasing the population of dermal microbes. Prohibiting or potentially reversing changes that lead to a run-down of skin repair, inflammation and acceleration of matrix breakdown.

### Hydration Failure

Hyaluronic acid and betaine, along with Olea europaea (olive) leaf extract, copper gluconate, quercetin and yeast beta-glucans aid the skins moisture levels. They have the technical effect of reversing the skins natural tendency to dry out as we get older. There are several reasons for the skin drying out, but the major cause is degradation of the skin's extracellular matrix. As the extracellular matrix degrades, the skin simply cannot hold as much fluid. The water retention, consequently hydration, can be boosted by re-building the extra-cellular matrix and providing hydrating agents. Each of hyaluronic acid, betaine, Olea europaea (olive) leaf extract, copper gluconate, quercetin and yeast beta-glucans have the technical effect of increasing hydration, aiding extracellular matrix, or both.

### Microcirculatory Collapse

We become more vulnerable to circulatory disease as we age. This applies equally to our skin. Olea europaea (olive) leaf extract and quercetin have a beneficial effect on the circulatory deterioration in the skin. They have the effect of improving the ability of our skin cells to gain the nutrients and oxygen they require. They can also benefit the skins processes for removing waste products. These compounds also stabilise the capillary beds in the skin in turn improving the microcirculation and repairing the skin.

### Immunological Rundown

There is convincing evidence that older skin is less immuno-competent. However, the yeast beta-glucans of the present invention has the beneficial effect of improving the skin's immunogenic response. This means that yeast beta-glucans can enhance the skin's own immune cells (such as Langerhans cells). There is an unexpected bonus; by stimulating (or priming) the Langerhans cells, the matrix-building cells (fibroblasts) are accentuated and start to build more of the extracellular matrix.

### Senescence

Senescence is the body's method of removing old cells. In some instances, cells remain past there functioning lifetime, using up resources and triggering chronic inflammation. This ages the skin dramatically. These cells are called senescent cells and need to be removed to rejuvenate the skin. Silybum marianum seed oil has the beneficial effect of removing these senescent cells that the body is no longer able to.

### Matrix Decay

The skin matrix is a very dynamic tissue and is very good at healing itself. The rate of matrix decay tends to accelerate, and the rate of matrix repair slows as we age, so the amount of matrix is progressively reduced. Inevitably dryness, sagginess and wrinkles start to form. Yeast beta-glucans and copper gluconate stimulate the Langerhans cells and the fibroblasts directly, so the rate of matrix generation speeds up. At the same time, the polyphenols quercetin and oleuropein slow the breakdown of the matrix. This one-two combo makes the matrix grow, so that the skin gains more elasticity, strength and hydration.

### Mitochondrial Decline

Ergothioneine provides an energy boost for your skin cells, which lose vitality as they age. This has the effect of rejuvenating your skin to make the skin look visibly younger.

### Epigenetic Drift

Betaine has a positive effect on epigenetic drift. Betaine can revert the skin cells to a younger state.

### EXAMPLES

### Example 1

A composition of the present invention was prepared according to the following formulation:

| Trade name | INCI | w/w % |
|---|---|---|
| RO water | Aqua | 47.05 |
| Solagum ™ AX | Acacia senegal gum | 0.5 |
| | Xanthan gum | |
| Zemea ® | Propanediol | 4 |
| SymSave ® H | Hydroxyacetophenone | 0.5 |
| Emulium ® Dolcea | Cetearyl alcohol | 4 |
| | Glyceryl stearate | |
| | Jojoba esters | |
| | Helianthus annuus (sunflower) seed wax | |
| | Sodium stearoyl glutamate | |
| | Water | |
| | Polyglycerin-3 | |
| | Tocopherol | |
| L22 ® | Jojoba oil/macadamia seed oil esters | 3 |
| | Squalene | |
| | Phytosteryl macadamiate | |
| | Phytosterols | |
| | Tocopherol | |
| Massocare SQV | Squalane | 2 |
| Jojoba Oil | Simmondsia chinensis (jojoba) seed oil | 2 |
| Stay-C ® 50 | Sodium ascorbyl phosphate | 2 |
| Genencare ® | Betaine | 2.5 |
| OSMS BA | | |
| EPS Seafill | Water | 2 |
| | Alteromonas ferment extract | |
| | (planktonic exopolysaccharide 3) | |
| | Phenetyl alcohol | |
| Rutin | Rutin | 0.05 |
| Lactil ® | Sodium lactate | 2 |
| | Sodium PCA | |
| | Glycine | |
| | Fructose | |
| | Urea | |
| | Niacinamide | |
| | Inositol | |
| | Lactic acid | |
| | Aqua | |
| | Sodium benzoate | |
| Telangyn ™ | Aqua | 2 |
| | Acetyl tetrapeptide-40 | |
| | Caprylyl glycol | |
| Oleuropein 80% | Olea europaea (olive) leaf extract | 1 |
| aurafirm P | Lactobacillus ferment | 2.5 |
| | Avena sativa kernel extract | |
| | Sodium benzoate | |
| | Potassium sorbate | |
| | Aqua | |
| LexFeel ™ | Heptyl undecylenate | 3 |
| WOW-A DT | C13-15 alkane | |
| SabiLize ® | Magnolia officinalis bark extract | 0.5 |
| | Thymus vulgaris (thyme) leaf oil | |
| | Glyceryl laurate | |
| Tocobiol ® sf | Tocopherol | 0.4 |
| Ephemer ™ | Caprylic/capric triglyceride | 1 |
| | Undaria pinnatifida extract | |
| Pro-Lipo ™ Neo | Propanediol | 3 |
| | Lecithin | |
| Milk thistle oil | Silybum marianum seed oil | 2 |
| Quercevita ™ | Lecithin | 0.5 |
| | Quercetin | |
| Vitamin A | Retinyl Palmitate | 0.35 |
| Palmitate/M 1.0 | | |
| MIU/g | | |
| Levagen ® | Palmitamide MEA | 0.3 |
| RO water | Aqua | 5 |
| Wellmune ® beta- | Yeast beta-glucan | 0.1 |
| glucan | | |
| Wheat germ | Triticum vulgare germ extract | 1 |
| extract/spermidine | | |
| MitoPrime ™ | Ergothioneine | 0.05 |
| Genencare ® | Betaine | 0.5 |
| OSMS BA | | |
| Zizania extract | Zizania latifolia stem extract | 0.2 |
| RO water | Aqua | 5 |

This composition is a liquid with a yellow/beige coloration. In certain embodiments, the first composition of the present invention may have a composition as per Example 1.

### Example 2

A composition of the present invention was prepared according to the following formulation:

| Trade name | INCI | w/w % |
|---|---|---|
| Emulium ® Dolcea | Cetearyl alcohol | 4 |
| | Glyceryl stearate | |
| | Jojoba esters | |
| | Helianthus annuus (sunflower) seed wax | |
| | Sodium stearoyl glutamate | |
| | Water | |
| | Polyglycerin-3 | |
| | Tocopherol | |
| Montanov ™ 68 | Cetearyl alcohol | 3 |
| | Cetearyl glucoside | |
| | Aqua | |
| | Glucose | |
| Lanette ® D | Cetearyl alcohol | 2 |
| L22 ® | Jojoba oil/macadamia seed oil esters | 5 |
| | Squalene | |
| | Phytosteryl macadamiate | |
| | Phytosterols | |
| | Tocopherol | |
| Massocare SQV | Squalane | 5 |
| Murumuru butter | Astrocaryum murumuru seed butter | 1 |
| Shea butter bio | Butyrospermum parkii butter | 3 |
| TimeCode ™ | Palmitoyl glycine | 1 |
| Cornflower water | Centaurea cyanus flower water | 30 |
| bio | | |
| Rheance ® One | Glycolipids | 1 |
| Genencare ® | Betaine | 2.5 |
| OSMS BA | | |
| SymSave ® H | Hydroxyacetophenone | 0.5 |
| Aquaxyl ™ | Xylitylglucoside | 3 |
| | Anhydroxylitol | |
| | Xylitol | |
| | Aqua | |
| | Glucose | |
| Solagum ™ AX | Acacia senegal gum | 0.3 |
| | Xanthan gum | |
| Sepitonic ™ M4 | Magnesium aspartate | 1 |
| | Zinc gluconate | |
| | Copper gluconate | |
| | Calcium gluconate | |
| SNAP-8 ™ | Acetyl octapeptide-3 | 3 |
| | Caprylyl glycol | |
| | Aqua | |
| Wonderage ™ | Aqua | 3 |
| | Propanediol | |
| | Glycerin | |
| | Cucurbitaceae extract | |
| | Pentylene glycol | |
| | Lactobacillus/brassica nigra | |
| | seed ferment extract | |
| | Lactococcus ferment extract | |
| Zemea ® | Propanediol | 4 |
| PrimalHyal ™ 50 | Hydrolysed hyaluronic acid | 0.5 |
| Tocobiol ® sf | Tocopherol | 0.4 |
| Bakuchiol | Bakuchiol | 1 |
| Dragosantol ® 100 | Bisabolol | 0.1 |
| LexFeel ™ | Heptyl undecylenate | 4 |
| WOW-A DT | C13-15 alkane | |
| EPS Seafill | Aqua | 2 |
| | Alteromonas ferment extract | |
| | (planktonic expolysaccharide 3) | |
| | Phenetyl alcohol | |
| SabiLize ® | Magnolia officinalis bark extract | 0.7 |
| | Thymus vulgaris (thyme) leaf oil | |
| | Glyceryl laurate | |
| Pro-Lipo ™ Neo | Propanediol | 4 |
| | Lecithin | |
| Milk thistle oil | Silybum marianum seed oil | 2 |
| Quercevita ™ | Lecithin | 0.5 |
| | Quercetin | |
| Vitamin A | Retinyl palmitate | 0.35 |
| Palmitate/M 1.0 | | |
| MIU/g | | |
| Levagen ® | Palmitamide MEA | 0.3 |
| RO water | Aqua | 5 |
| Wellmune ® beta- | Yeast beta-glucan | 0.1 |
| glucan | | |
| Wheat germ | Triticum vulgare germ extract | 1 |
| extract/spermidine | | |
| MitoPrime ™ | Ergothioneine | 0.05 |
| Genencare ® | Betaine | 0.5 |
| OSMS BA | | |
| Zizania extract | Zizania latifolia stem extract | 0.2 |
| RO water | Aqua | 5 |

This composition is a cream. In certain embodiments, the second composition of the present invention may have a composition as per Example 2.

### Example 3

A composition of the present invention was prepared according to the following formulation:

| | | First | Second | Protocol |
|---|---|---|---|---|
| | | Composition | Composition | Dose |
| Trade Name | INCI Name | Dose (w/w %) | Dose (w/w %) | (w/w %) |
| RO Water | Aqua | 57.05 | 10 | 67.05 |
| Solagum ™ AX | Acacia senegal gum | 0.5 | 0.3 | 0.8 |
| | Xanthan gum | | | |
| Zemea ® | Propanediol | 4 | 4 | 8 |
| SymSave ® H | Hydroxyacetophenone | 0.5 | 0.5 | 1 |
| Emulium ® Dolcea | Cetearyl alcohol | 4 | 4 | 8 |
| | Glyceryl stearate | | | |
| | Jojoba esters | | | |
| | Helianthus annuus | | | |
| | (sunflower) seed wax | | | |
| | Sodium stearoyl | | | |
| | glutamate | | | |
| | Water | | | |
| | Polyglycerin-3 | | | |
| | Tocopherol | | | |
| L22 ® | Jojoba oil/macadamia | 3 | 5 | 8 |
| | seed oil esters | | | |
| | Squalene | | | |
| | Phytosteryl | | | |
| | macadamiate | | | |
| | Phytosterols | | | |
| | Tocopherol | | | |
| Massocare SQV | Squalane | 2 | 5 | 7 |
| Jojoba oil | Simmondsia chinensis | 2 | 0 | 2 |
| | (jojoba) seed oil | | | |
| Stay-C ® 50 | Sodium ascorbyl | 2 | 0 | 2 |
| | phosphate | | | |
| Genencare ® OSMS BA | Betaine | 3 | 3 | 6 |
| EPS Seafill | Water | 2 | 2 | 4 |
| | Alteromonas ferment | | | |
| | extract (planktoic | | | |
| | exopolysaccharide 3) | | | |
| | Phenetyl alcohol | | | |
| Rutin | Rutin | 0.05 | 0 | 0.05 |
| Lactil ® | Sodium lactate | 2 | 0 | 2 |
| | Sodium PCA | | | |
| | Glycine | | | |
| | Fructose | | | |
| | Urea | | | |
| | Niacinamide | | | |
| | Inositol | | | |
| | Lactic acid | | | |
| | Aqua | | | |
| | Sodium benzoate | | | |
| Telangyn ™ | Aqua | 2 | 0 | 2 |
| | Acetyl tetrapeptide-40 | | | |
| | Capylyl glycol | | | |
| Oleuropein 80% | Olea europaea (olive) | 1 | 0 | 1 |
| | leaf extract | | | |
| aurafirm P | Lactobacillus ferment | 2.5 | 0 | 2.5 |
| | Avena sativa kernel | | | |
| | extract | | | |
| | Sodium benzoate | | | |
| | Potassium sorbate | | | |
| | Aqua | | | |
| LexFeel ™ WOW-A | Heptyl undecylenate | 3 | 4 | 7 |
| DT | C13-15 alkane | | | |
| SabiLize ® | Magnolia officinalis bark | 0.5 | 0.7 | 1.2 |
| | extract | | | |
| | Thymus vulgaris (thyme) | | | |
| | leaf oil | | | |
| | Glyceryl laurate | | | |
| Tocobiol ® sf | Tocopherol | 0.4 | 0.4 | 0.8 |
| Ephemer ™ | Caprylic/capric | 1 | 0 | 1 |
| | triglyceride | | | |
| | Undaria pinnatifida | | | |
| | extract | | | |
| Pro-Lipo ™ Neo | Propanediol | 3 | 4 | 7 |
| | Lecithin | | | |
| Milk thistle oil | Silybum marianum seed oil | 2 | 2 | 4 |
| Quercevita ™ | Lecithin | 0.5 | 0.5 | 1 |
| | Quercetin | | | |
| Vitamin A | Retinyl palmitate | 0.35 | 0.35 | 0.7 |
| Palmitate/M 1.0 | | | | |
| MIU/g | | | | |
| Levagen ® | Palmitamide MEA | 0.3 | 0.3 | 0.6 |
| Wellmune ® Beta- | Yeast beta-glucan | 0.1 | 0.1 | 0.2 |
| Glucan | | | | |
| Wheat germ | Triticum vulgare germ | 1 | 1 | 2 |
| extract/spermidine | extract | | | |
| MitoPrime ™ | Ergothioneine | 0.05 | 0.05 | 0.1 |
| Zizania Extract | Zizania latifolia stem | 0.2 | 0.2 | 0.4 |
| | extract | | | |
| Montanov ™ 68 | Cetearyl alcohol | 0 | 3 | 3 |
| | Cetearyl glucoside | | | |
| | Aqua | | | |
| | Glucose | | | |
| Lanette ® D | Cetearyl alcohol | 0 | 2 | 2 |
| Murumuru butter | Astrocaryum murumuru | 0 | 1 | 1 |
| | seed butter | | | |
| Shea butter bio | Butyrospermum parkii | 0 | 3 | 3 |
| | butter | | | |
| TimeCode ™ | Palmitoyl glycine | 0 | 1 | 1 |
| Cornflower water | Centaurea cyanus flower | 0 | 30 | 30 |
| bio | water | | | |
| Rheance ® One | Glycolipids | 0 | 1 | 1 |
| Aquaxyl ™ | Xylitylglucoside | 0 | 3 | 3 |
| | Anhydroxylitol | | | |
| | Xylitol | | | |
| | Aqua | | | |
| | Glucose | | | |
| Sepitonic ™ M4 | Magnesium aspartate | 0 | 1 | 1 |
| | Zinc gluconate | | | |
| | Copper gluconate | | | |
| | Calcium gluconate | | | |
| SNAP-8 ™ | Acetyl octapeptide-3 | 0 | 3 | 3 |
| | Caprylyl glycol | | | |
| | Aqua | | | |
| Wonderage ™ | Aqua | 0 | 3 | 3 |
| | Propanediol | | | |
| | Glycerin | | | |
| | Cucurbitaceae extract | | | |
| | Pentylene glycol | | | |
| | Lactobacillus/brassica | | | |
| | nigra seed ferment | | | |
| | extract | | | |
| | Lactococcus ferment | | | |
| | extract | | | |
| PrimalHyal ™ 50 | Hydrolysed hyaluronic | 0 | 0.5 | 0.5 |
| | acid | | | |
| Bakuchiol | Bakuchiol | 0 | 1 | 1 |
| Dragosantol ® 100 | Bisabolol | 0 | 0.1 | 0.1 |

Liposomes are prepared by mixing propanediol, lecithin, tocopherol and Helianthus annuus (sunflower seed oil). The liposomes are then loaded with betaine (which was dissolved at 40° C. in water or milk thistle oil), Silybum marianum seed oil, ergothioneine (which was dissolved at 40° C. in water or milk thistle oil), Triticum vulgare germ extract, palmitamide MEA, quercetin, retinyl palmitate, yeast beta-glucan (which was dissolved at 40° C. in water or milk thistle oil), and Zizania latifolia stem extract (which was dissolved at 40° C. in water or milk thistle oil) by stirring the liposomes with these components at a speed that will not disrupt the liposomes.

The components not forming part of the liposomes are mixed together in the form they are supplied in the amounts set out above. Except that Levagen® is dissolved in water or milk thistle oil at a temperature of 90° C. The liposome mixture is also combined with the other ingredients.

### Example 4

A composition of Example 3 was administered to 30 subjects for 30 days, after which they were subjected to industry standard testing including skin thickness, tensile strength, smoothness and visco-elasticity.

All parameters measured, skin thickness, tensile strength, smoothness and visco-elasticity, showed significant improvements of between 30 and 50% over baseline.

### Example 5

To ensure product stability, a composition within packaging was subjected to a temperature cycle test by putting the products into a freezer at −16° C. and then heating the product to 40° C.

This cycle was repeated five times to see if there were any changes in stability to the products.

Emulsifier stability was shown to be low when in combination. However, a composition comprising thyme oil provided a stable composition.

The test was conducted on a composition of Example 1 where the SabiLize® component of Example 2 was not present and the 0.5 wt % of SabiLize® was replaced with aqua. This will be referred to as Comparative Example 1 The test was also conducted on a composition of Example 2 where the SabiLize® component of Example 2 was not present and the 0.7 wt % of SabiLize® was replaced with Centaurea cyanus flower water. This will be referred to as Comparative Example 2.

Example 1 possessed the following characteristics when in the packaging prior to the testing.
Appearance: Homogeneous cream
Colour: Greenish brown
Odour: characteristic for raw materials used
pH: 5.0-6.0

Example 2 possessed the following characteristics when in the packaging prior to testing.
Appearance: Homogeneous cream
Colour: yellow
Odour: characteristic for raw materials used
pH: 4.8-5.5

The stability of Example 1, Example 2, Comparative Example 1, and Comparative Example 2 was tested with a Cycle test where the temperature of the composition within the packaging was held at −16° C. for 24 hours and then heated to 40° C.) and held at that temperature for 24 hours. The product was then cooled again to repeat the cool and heat cycle for a total of three times. The pH was tested before the start of the stability test and at the end of temperature cycle.

### Results—Composition 1

| | | Cycle test | | |
|---|---|---|---|---|
| Parameters | Description | First cycle | Second cycle | Third cycle |
| Appearance | Cream | No changes | No changes | No changes |
| Colour | Greenish | No changes | Brownish | Brownish |
| | brown | | | |
| Odour | Characteristic | No changes | No changes | No changes |
| | for raw | | | |
| | materials used | | | |
| pH* | 5.4 | 5.3 | 5.4 | 5.25 |

### Results—Composition 2

| | | Cycle test | | |
|---|---|---|---|---|
| Parameters | Description | First cycle | Second cycle | Third cycle |
| Appearance | Cream | No changes | No changes | No changes |
| Colour | Yellowish | No changes | Brownish | Brownish |
| Odour | Characteristic | No changes | No changes | No changes |
| | for raw | | | |
| | materials used | | | |
| pH* | 5.08 | 5.1 | 5.2 | 5.4 |

Both Comparative Example 1 and Comparative Example 2 showed signs of instability during the test protocol.

### Conclusion

After conducting the cosmetic product stability in 3 cycles in the original packaging, the organoleptic and pH test results were stable. The differences in the pH results are insignificant and are within acceptable range of measurement uncertainty adopted for the method. Colour change is normal due to the natural extracts. We did not observe any changes in the appearance of the packaging. Taking into account the above, Composition 1 and Composition 2 remain stable and were compatible with their packaging under fluctuating temperature conditions during the total duration of the cycle test.

### Example 6

Background and aims: The clinical trial of Example 6 aimed to determine changes in the appearance and texture of skin after the application of the composition of Example 3, utilising both the first composition and the second composition to provide the protocol dose.

Methods: A total of 50 participants were included in the trial. Age criteria of participants was between 30-65 years of age. The parameters measured to determine changes to skin were hydration, elasticity and redness. Measurements were taken at baseline and at the end of the 12 week period, in which the protocol was applied to the facial skin twice per day, every day.

Results: Skin hydration was improved by an average of 72%. Skin elasticity was improved by an average of 24%. Skin redness was reduced by an average of 21%.

Conclusion: The skincare protocol of Example 3 improved the appearance and texture of skin and resulted in anti-aging effects. The protocol improved all parameters measured, including hydration, elasticity and redness.

### Aims

This clinical trial aims to establish the benefits of the protocol of Example 3 on skin hydration, elasticity and redness. This is the first protocol that has been able to stabilise the high number of active ingredients within it allowing it to address all mechanisms of skin aging.

### Methods

### Study Population

A total of 50 participants were included in the trial. Age criteria of participants was between 30-65 years of age, with the average age of the population being 49 years old. 43 of the participants were female and 7 participants were male.

### Exclusion Criteria

Participants were excluded from taking part in the trial if they had any surgery or invasive treatment on their skin which affected skin appearance or condition, including botox or fillers injected into the skin. Participants with diagnosed skin conditions or disease were also excluded.

### Measurement of Skin Parameters

The parameters measured to determine changes to skin were hydration, elasticity and redness. Skin elasticity was measured using Cutometer Dual MPA 580 device, skin hydration was measured using the Corneometer CM 825 device and skin redness was measured using Colorimeter CL-400 device. These devices were chosen as previous studies have demonstrated the reliability of results obtained from them, validating them for purpose.

Measurements were taken at baseline and at the end of the 12 week period, in which the protocol was applied to the facial skin twice per day, every day.

### Statistical Analysis

Data analysis was performed using the Statistical Package for the Social Sciences (SPSS). The Shapiro-Wilk test was used to determine whether or not data was normally distributed—when P>0.05, data was considered to be normally distributed. Parametric tests for significance were carried out on normally distributed data and non-parametric tests for significance were carried out on data which was not normally distributed.

The parametric test for significance used was the paired samples t-test and the non-parametric test for significance used was the Wilcoxon signed-rank test. These tests were conducted in order to establish any significant differences between the results for hydration, elasticity and redness before and after the treatment. When P<0.05, differences were considered statistically significant.

### Results

There was a total of 50 participants included in the study. The average age of the population was 49 years old. Table 1 shows the average baseline and endpoint scores for each parameter measured. All differences were found to be statistically significant (P<0.05).

TABLE 1
Average baseline and endpoint scores
for hydration, elasticity and redness

| | Baseline Mean (AU) | Endpoint Mean (AU) | P-value |
|---|---|---|---|
| Hydration | 44.6 | 76.6 | <0.001 |
| | Baseline Mean (R2) | Endpoint Mean (R2) | P-value |
| Elasticity | 0.45 | 0.56 | <0.001 |
| | Baseline Mean (ITA) | Endpoint Mean (ITA) | P-value |
| Redness | 41.7 | 50.6 | <0.001 |

Improvement was seen for all parameters measured in all participants included in the trial. Table 2 displays the minimum, maximum and average improvement in hydration, elasticity and redness.

TABLE 2
Improvements in skin parameters measured

| | Number of | | | |
|---|---|---|---|---|
| | participants | | | |
| | with | Minimum | Maximum | Average |
| | improvement | improvement | improvement | improvement |
| | (%) | (%) | (%) | (%) |
| Hydration | 100 | 65 | 81 | 72 |
| Elasticity | 100 | 18 | 31 | 24 |
| Redness | 100 | 17 | 29 | 21 |

### Conclusion

The findings of this clinical trial indicate that the protocol of Example 3 is effective at increasing the hydration status and elasticity of skin and reducing the appearance of redness. The protocol is highly efficient at restoring skin hydration—a lack of which is a major contributor to the appearance of aging skin, including dullness and visible wrinkles.

No adverse effects were recorded throughout the duration of the trial.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A composition comprising retinyl palmitate, Silybum marianum seed oil, betaine, ergothioneine, yeast beta-glucan, Triticum vulgare germ extract, quercetin, and propanediol.

2. The composition of claim 1, wherein the composition comprises retinyl palmitate, milk thistle oil, Genencare® OSMS BA, MitoPrime™, Wellmune®, Triticum vulgare germ extract, Quercevita™, and Pro-Lipo™ Neo and/or Zemea®.

3. The composition of claim 1, wherein the retinyl palmitate is present in an amount of from about 0.25 to about 0.5 wt %, optionally in an amount of about 0.35 wt %.

4. The composition of claim 1, wherein the Silybum marianum seed oil (milk thistle oil) is present in an amount of from about 1 to about 3 wt %, optionally the Silybum marianum seed oil is present in an amount of about 2 wt %.

5. The composition of claim 1, wherein the betaine is present in an amount of from about 2 to about 4 wt %, optionally the betaine is present in an amount of about 2.5 wt %.

6. The composition of claim 1, wherein the ergothioneine is present in an amount of from about 0.01 to about 0.1 wt %, optionally the ergothioneine is present in an amount of about 0.05 wt %.

7. The composition of claim 1, wherein the yeast beta-glucan is present in an amount of from about 0.05 to about 0.3 wt %, optionally the yeast beta-glucan is present in an amount of about 0.1 wt %.

8. The composition of claim 1, wherein the quercetin is present in an amount of from about 0.002 to about 0.4 wt %, optionally the quercetin is present in an amount of about 0.1 wt %.

9. The composition of claim 1, wherein the propanediol is present in an amount of from about 1 to about 8 wt % or from about 3 to 8 wt %, optionally the propanediol is present in an amount of about 5.5 or about 6.2 wt %.

10. The composition of any-r-eeedig-claim 1, further comprising one or more components selected from: Jojoba oil, macadamia seed oil esters, squalene, phytosteryl macadamiate, phytosterols (L22), cetearyl alcohol, glyceryl stearate, jojoba esters, Helianthus annus (sunflower) seed wax, sodium stearoyl glutamate, polyglycerin-3 (Emulium Dolcea®), palmitamide MEA (Levagen®), phenetyl alcohol, planktonic exopolysaccharide 3 (EPS Seafill), tocopherol, Zizania latifolia stem extract, lecithin, (Pro-Lipo™ Neo) heptyl undecylenate, and C₁₃₋₁₅ alkane (LexFeel™ WOW-A DT).

11. The composition of claim 1, further comprising one or more components selected from: sodium ascorbyl phosphate (Stay-C® 50), Simmondsia chinensis (jojoba) seed oil, Olea europaea (olive) leaf extract, sodium lactate, Sodium pyrrolidone carboxylic acid, fructose, glycine, inositol, niacinamide, urea, sodium benzoate, lactic acid (Lactil®), Avena sativa kernel extract, Lactobacillus ferment (aurafirm), Acetyl tetrapeptide-40, Caprylyl glycol (Telangyn™), rutin, and Undaria pinnatifida extract (Ephemer™).

12. The composition of claim 1, further comprising one or more components selected from: Centaurea cyanus flower water, Butyrospermum parkii butter, Xylitylglucoside, Anhydroxylitol, Xylitol (Aquaxyl™) Astrocaryum murumuru seed butter, bakuchiol, Palmitoyl glycine (Timecode™), Acetyl octapeptide-3, Magnesium aspartate, Zinc gluconate, Copper gluconate (Sepitonic™ M4), Glycerin, Cucurbitaceae (gourd) extract, Lactobacillus/brassica nigra seed ferment extract, lactococcus ferment extract, Pentylene glycol (Wonderage™), Hydrolysed hyaluronic acid (PrimaHyal™ 50), Bisabolol and cetearyl alcohol, cetearyl glucoside, glucose (Montanov™ 68).

13. The composition of claim 1, wherein the composition is a two-part composition with a first composition and a second composition, wherein:
the first composition comprises retinyl palmitate, Silybum marianum seed oil, betaine, ergothioneine, yeast beta-glucan, Triticum vulgare germ extract, quercetin, and propanediol; and
the second composition comprises retinyl palmitate, Silybum marianum seed oil, betaine, ergothioneine, yeast beta-glucan, Triticum vulgare germ extract, quercetin, and propanediol.

14. The composition of claim 13, wherein the first composition and the second composition have different viscosities, optionally the first composition has a lower viscosity than the second composition.

15. The composition of claim 1, wherein the composition further comprises thyme oil.

16. A skin anti-ageing protocol wherein the protocol is a method of cosmetic therapy comprising application to a user's skin of:
a first composition comprising retinyl palmitate, Silybum marianum seed oil, betaine, ergothioneine, yeast beta-glucan, Triticum vulgare germ extract, quercetin, and propanediol; and
a second composition comprising retinyl palmitate, Silybum marianum seed oil, betaine, ergothioneine, yeast beta-glucan, Triticum vulgare germ extract, quercetin, and propanediol.

17. The skin anti-ageing protocol of claim 16, wherein the first composition is a serum and the second composition is a cream.

18. A kit comprising a first composition of claim 1 and a second composition of claim 1.

19. The kit of claim 18, further comprising instructions on how to apply the first composition and the second composition.
